(19) United States
(12) Patent Application Publication
(10) Pub. No.: US 2022/0218532 A1
(43) Pub. Date: Jul. 14, 2022

(54) Release Liner For Simultaneous Use With Two Adhesives

(71) Applicant: KCI Licensing, Inc., San Antonio, TX (US)

(72) Inventors: James A. LUCKEMEYER, San Antonio, TX (US); Timothy Mark ROBINSON, Shillingstone (GB); Christopher Brian LOCKE, Bournemouth (GB)

(21) Appl. No.: 17/607,775

(22) PCT Filed: Mar. 20, 2020
(86) PCT No.: PCT/US2020/023848

§ 371 Date: Oct. 29, 2021

Related U.S. Application Data
(60) Provisional application No. 62/849,268, filed on May 17, 2019.

(51) Int. Cl. A61F 13/02; A61F 13/00; A61F 13/512

(52) U.S. Cl. CPC A61F 13/0266; A61F 13/00068; A61F 13/0216; A61F 2013/00604; A61F 13/0253; A61F 2013/00702; A61F 2013/00663; A61F 13/512

(57) ABSTRACT

A release liner, for example for use with a tissue cover having a first adhesive and a second adhesive, may comprise a first release agent adapted for the first adhesive and a second release agent adapted for the second adhesive. Typically, the first release agent is different from the second release agent, and the first release agent may be configured to interact with a first area of the cover having the first adhesive, and the second release agent may be configured to interact with a second area of the cover having the second adhesive. In some embodiments, the release liner may be multi-layered, for example with a first layer comprising the first release agent and a second layer comprising the second release agent. Some such embodiments may have perforations through the second layer of the release liner, with the first layer then being disposed with respect to the second layer in such a way as to expose the first release agent through the perforations in the second layer.

### RELATED APPLICATION

The present invention claims the benefit of the filing of U.S. Provisional Patent Application No. 62/849,268, filed May 17, 2019, which is incorporated herein by reference for all purposes.

### TECHNICAL FIELD

The invention set forth in the appended claims relates generally to tissue treatment systems and more particularly, but without limitation, to systems, apparatuses, and methods for covering tissues sites and/or for protecting adhesives for such tissue coverings until ready for application to the tissue site.

### BACKGROUND

Dressings are generally considered standard care for many types of tissue treatment, particularly for treating wounds. Regardless of the etiology of a wound, whether trauma, surgery, or another cause, proper care of the wound is important to the outcome. Dressings can provide many functions that can be beneficial for healing wounds, including controlling the wound environment and protecting a wound from bacteria and further physical trauma.

While the benefits of dressings are widely known, improvements to dressing technology can benefit healthcare providers and patients.

### BRIEF SUMMARY

New and useful systems, apparatuses, and methods for treating tissues are set forth in the appended claims. Illustrative embodiments are also provided to enable a person skilled in the art to make and use the claimed subject matter.

For example, in some embodiments, a release liner may be configured to operate effectively on a tissue cover with two adhesives, with the release liner comprising or consisting essentially of two release agents. Typically, a first release agent may be configured, adapted, or selected for effective interaction with a first adhesive of the cover, while the second release agent may be configured, adapted, or selected for effective interaction with a second adhesive of the cover. For example, a release liner may be configured for use with a cover having both acrylic adhesive and silicone adhesive, with the first release agent configured for the acrylic adhesive and the second release agent configured for the silicone adhesive. In some embodiments, the release liner may comprise two layers, for example with a first layer having the first release agent and the second layer having the second release agent. In some examples, one of the layers may have perforations or apertures, and the layers may be disposed or configured so that one of the release agents is exposed through the perforations or apertures. For example, if the second layer has perforations and the second release agent is exposed on its surface, then the first layer may typically be located adjacent to the second layer so that the first release agent may be exposed through the perforations or apertures in the second layer. In this way, a release liner may be configured to allow simultaneous interaction of the first release agent and the second release agent.

More generally, in some embodiments a release liner for protecting a first adhesive and a second adhesive may comprise a carrier; a first release agent disposed on the carrier and adapted for the first adhesive; and a second release agent disposed on the carrier and adapted for the second adhesive. In some embodiments of the release liner, the carrier may comprise a first layer carrier and a second layer carrier, for example with the first release agent disposed on the first layer carrier and the second release agent disposed on the second layer carrier. In some embodiments, the second layer carrier and the second release agent may comprise a plurality of perforations, and the second layer carrier may be disposed adjacent to the first release agent such that the first release agent is exposed through the plurality of perforations.

Some example embodiments may comprise a release liner for use with a tissue cover having a first adhesive and a second adhesive, wherein the first and second adhesives are different from each other, the release liner comprising: a first release agent adapted for the first adhesive; and a second release agent adapted for the second adhesive. Typically, the first release agent may be different from the second release agent. In some embodiments, the first release agent may be configured to interact with a first area of the cover having the first adhesive, and the second release agent may be configured to interact with a second area of the cover having the second adhesive. The release liner may also comprise a first layer and a second layer; for example, the first layer may comprise the first release agent and the second layer may comprise the second release agent. In some embodiments, the first layer of the release liner may further comprise a first carrier and/or the second layer of the release liner may further comprise a second carrier.

Cover embodiments are also described herein, wherein some example embodiments may comprise: a first area having a first peel strength; a second area having a second peel strength; and a release liner comprising a first release agent adjacent to the first area and a second release agent adjacent to the second area. Some embodiments of a cover may comprise: a shell layer having an adhesive coating; a contact layer adjacent to the shell layer, the contact layer comprising a first plurality of apertures; and a release liner comprising: a film; a first release layer disposed adjacent to the contact layer, the first release layer comprising a first release agent and a second plurality of apertures through which at least some of the adhesive coating is exposed; and a second release layer disposed adjacent to the first release layer, the second release layer comprising a second release agent disposed adjacent to the adhesive coating exposed through the second plurality of apertures.

In some embodiments, a cover may comprise a first area having a first adhesive and/or a first peel strength; a second area having a second adhesive and/or second peel strength; and a release liner having a first release agent and a second release agent. Typically, the first adhesive may differ from the second adhesive, and the first release agent may differ from the second release agent. In some instances, the first peel strength may be greater than the second peel strength. In some embodiments the first release agent may be adjacent to and/or positioned for interaction with the first area, while the second release agent may be adjacent to and/or positioned for interaction with the second area. In some embodiments, the second area may be located on a layer of the cover 100 having apertures therethrough.

In some exemplary embodiments, the first release agent may be disposed on a first layer, the second release agent may be disposed on a second layer, the first area may be disposed on a third layer, and the second area may be disposed on a fourth layer which comprises a plurality of apertures. For example, the third layer may be a shell layer, and the fourth layer may be a contact layer. In some embodiments, a pattern of a plurality of apertures on the fourth layer may match, register with, and/or align with a pattern of perforations of the second layer of the release liner. For example, the centerline of each aperture in the fourth layer may be substantially aligned with the centerline of the corresponding perforation of the second layer of the release liner. In some embodiments, the first release agent may be disposed in stacked proximity to the first area, and the second release agent may be disposed in stacked proximity to the second area. For example, in some embodiments the first release agent may be disposed in stacked proximity to the first area through the aligned perforations in the second layer of the release liner and apertures in the contact layer. In some embodiments, the contact layer may be located between the shell layer and the second layer of the release liner, such that the first release agent may be configured to interact substantially with the first area and the second release agent may be configured to interact substantially with the second area.

In some embodiments, the first area may comprise acrylic adhesive, and the second area may comprise silicone. The area of silicone may be positioned or disposed with respect to the area of acrylic adhesive so as to expose the first area of acrylic adhesive through the apertures. In such embodiments, the first release agent may have a release factor configured to facilitate manual release from acrylic adhesive, and the second release agent may have a release factor configured to facilitate manual release from silicone.

A method of manufacturing a release liner is also described herein, with some exemplary embodiments comprising: providing a first layer comprising a first release agent adapted for a first adhesive; providing a second layer comprising a second release agent adapted for a second adhesive, wherein the second layer comprises a plurality of perforations; disposing the first layer adjacent with the second layer; wherein the first release agent is exposed through the plurality of perforations in the second layer.

A method of manufacturing a cover for a tissue site is also described herein, with some exemplary embodiments comprising one or more of the following steps: providing a shell layer comprising a first adhesive; providing a contact layer comprising a second adhesive; providing a first layer of release liner comprising a first release agent adapted for the first adhesive; providing a second layer of release liner comprising a second release agent adapted for the second adhesive; applying the second layer of release liner to the contact layer; perforating the contact layer and the second layer of release liner to form a plurality of perforations; applying the shell layer to the contact layer opposite the second layer of release liner; and/or applying the first layer of release liner to the second layer of release liner opposite the contact layer. In some embodiments, the first adhesive may comprise acrylic, while the second adhesive may comprise silicone. In some embodiments, applying the second layer of release liner to the contact layer may comprise stacking the second layer with the contact layer, so that the second release agent is adjacent to the second adhesive.

In some embodiments, applying the shell layer to the contact layer opposite the second layer of release liner may include disposing the shell layer with the first adhesive facing and/or contacting the contact layer, such that the first adhesive is exposed through the plurality of perforations/apertures in the contact layer. In some embodiments, applying the first layer of release liner to the second layer of release liner opposite the contact layer may result in the first release agent being exposed through the perforations in the contact layer and the second layer of release liner to the first adhesive. In some embodiments, perforating the contact layer and the second layer of the release liner may occur simultaneously, for example while they are joined in stacked, parallel plane relationship.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is an assembly view of an exemplary cover that can be applied to a tissue site;

FIG. 2 is a top view of an example of a contact layer that may be associated with some embodiments of the cover of FIG. 1;

FIG. 3 is a detail view of the contact layer of FIG. 2;

FIG. 4 is an isometric view of an example of the cover of FIG. 1, illustrating additional details that may be associated with some embodiments;

FIG. 5 is an assembly view of an exemplary release liner that may be associated with some embodiments of a cover, such as the cover of FIG. 1;

FIG. 6 is an elevation view of the release liner of FIG. 5;

FIG. 7 is a schematic cross-section view of the release liner of FIG. 6;

FIG. 8 is a plan view of another release liner embodiment that may be associated with some embodiments of a cover, such as the cover of FIG. 1;

FIG. 9 is a schematic view of an embodiment related to the release liner of FIG. 8;

FIG. 10 is a schematic view of another embodiment related to the release liner of FIG. 8;

FIG. 11 is a detail cross-section view of the cover of FIG. 1, with an exemplary release liner similar to that shown in FIG. 5;

FIG. 12 is a schematic diagram illustrating an example of the cover of FIG. 1 used with a therapy system that can provide negative-pressure treatment to a tissue site; and

FIG. 13 is a detail view of the cover of FIG. 12.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

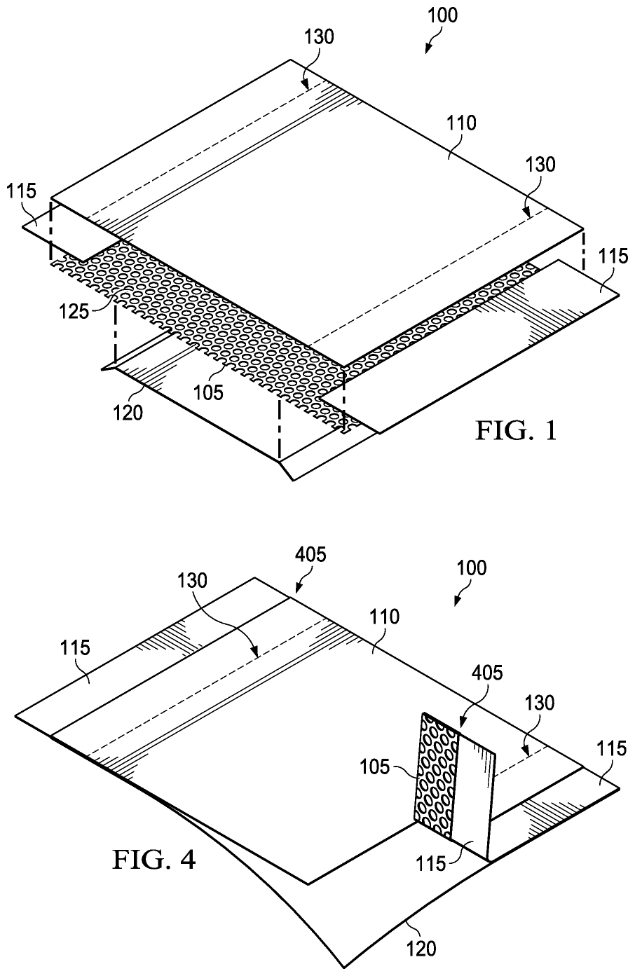

FIG. 1 is an assembly view of an example of a cover 100, which can be applied to a tissue site. The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, a tissue site may be used to grow additional tissue that can be harvested and transplanted.

The cover 100 of FIG. 1 generally includes a contact layer 105 and a shell layer 110. As illustrated in the example of FIG. 1, the cover 100 may additionally include one or more handling bars 115 and a release liner 120. As illustrated in the example of FIG. 1, the contact layer 105 and the shell layer 110 may be perforated. For example, the contact layer 105 of FIG. 1 has a plurality of apertures 125, and the shell layer 110 has a plurality of apertures 130. The apertures 125 may form passageways through the thickness of the contact layer 105 in some examples.

In some embodiments, the contact layer 105 may comprise or consist essentially of a soft, pliable material suitable for contact with the patient's skin. The contact layer 105 may also have an adherent or tacky surface. Thus, the contact layer may comprise an adhesive and/or consist essentially of a material with an adhesive quality that may serve as an adhesive. For example, the contact layer 105 may comprise or consist essentially of a tacky gel having a peel adhesion of about 0.2 to 0.3 N/cm (180 degree peel on stainless steel). The contact layer 105 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In embodiments comprising or consisting essentially of silicone gel or soft silicone, the silicone gel or soft silicone may serve as an adhesive by presenting a tacky surface. In some embodiments, the contact layer 105 may have a thickness between about 200 microns (μm) and about 1000 microns (μm). In some embodiments, the contact layer 105 may have a hardness between about 5 Shore OO and about 80 Shore OO. Further, the contact layer 105 may be comprised of hydrophobic or hydrophilic materials.

In some embodiments, the contact layer 105 may be a coated material. For example, the contact layer 105 may be formed by coating a porous material, such as, for example, a woven, a nonwoven, or an extruded mesh with a hydrophobic material. The hydrophobic material for the coating may be a soft silicone, for example.

In some embodiments, the shell layer 110 may provide a bacterial barrier and protection from physical trauma. The shell layer 110 may also be constructed from a material that can reduce evaporative losses and provide a fluid seal between two components or two environments, such as between a therapeutic environment and a local external environment. The shell layer 110 may comprise or consist of, for example, an elastomeric film or membrane that can provide a seal adequate to maintain a negative pressure at a tissue site for a given negative-pressure source. The shell layer 110 may have a high moisture-vapor transmission rate (MVTR) in some applications. For example, the MVTR may be at least 250 grams per square meter per twenty-four hours (g/m2/24 hours) in some embodiments, measured using an upright cup technique according to ASTM E96/E96M Upright Cup Method at 38° C. and 10% relative humidity (RH). In some embodiments, an MVTR up to 5,000 g/m2/24 hours may provide effective breathability and mechanical properties.

In some example embodiments, the shell layer 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The shell layer 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis, Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inspire 2327 polyurethane films, commercially available from TC Transcontinental, Wrexham, United Kingdom. In some embodiments, the shell layer 110 may comprise Inspire 2301 having an MVTR (upright cup technique) of 2600 g/m2/24 hours and a thickness of about 30 microns.

An attachment device may be disposed on a side of the shell layer 110. The attachment device may take many forms. For example, an attachment device may be a medically-acceptable, pressure-sensitive adhesive disposed on a side of the shell layer 110 facing the contact layer 105. At least some of the adhesive may be disposed adjacent to the apertures 125. In some embodiments, for example, some or all of one side of the shell layer 110 may be coated with an adhesive, such as an acrylic adhesive, which may have a coating weight of about 25-65 grams per square meter (g.s.m.). Other example embodiments of an attachment device may include a double-sided tape, paste, hydrocolloid, hydrogel, silicone gel, or organogel.

In some configurations, additional layers (not shown) may be disposed between the shell layer 110 and the contact layer 105. For example, a scrim layer may be used with an adhesive to facilitate manufacture, or an absorbent may be disposed between portions of the contact layer 105 and the shell layer 110.

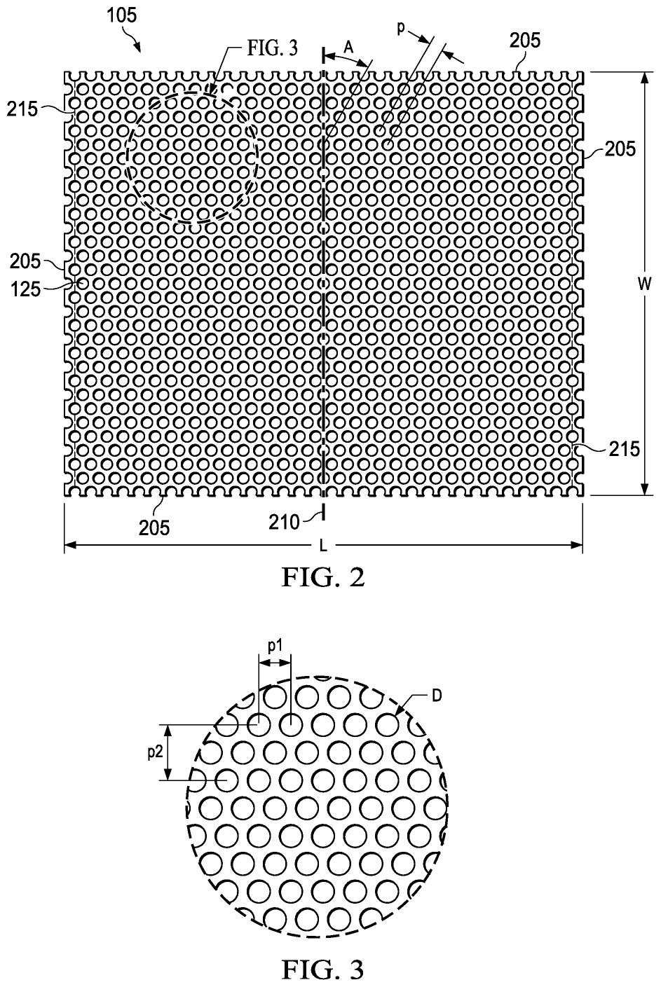

FIG. 2 is a top view of an example of the contact layer 105, illustrating additional details that may be associated with some embodiments. FIG. 3 is a detail view of the contact layer 105 in the example of FIG. 2, illustrating additional details that may be associated with some embodiments. In the example of FIG. 2, the contact layer 105 is rectangular, having edges 205, a width W, and a length L. The apertures 125 may be characterized by various properties, such as hole shape, hole size, hole pattern, and pattern orientation.

The apertures 125 may have many shapes, including circles, squares, stars, ovals, polygons, slits, complex curves, rectilinear shapes, triangles, or some combination of such shapes.

The size of the apertures 125 may be specified by a single dimension in some examples, such as a width of a circle or a square. In some examples, the size may be specified by a length (the longer of two dimensions) and width (the shorter of two dimensions). In some embodiments, each of the apertures 125 may have a width of about 1 millimeter to about 50 millimeters. A width of about 6 millimeters to about 8 millimeters may be suitable for some embodiments. Each of the apertures 125 may have uniform or similar sizes. For example, in some embodiments, each of the apertures 125 may have substantially the same width. In other embodiments, geometric properties of the apertures 125 may vary. For example, the width of the apertures 125 may vary depending on the position of the apertures 125 in the contact layer 105. In some embodiments, the width of the apertures 125 may be larger in a peripheral area than an interior area of the contact layer 105. At least some of the apertures 125 may be positioned on one or more of the edges 205 of the contact layer 105, and may have an interior cut open or exposed at the edges 205.

The apertures 125 may be arranged in a pattern. For example, the apertures 125 may have a uniform distribution pattern, such as an arrangement of rows, or may be randomly distributed in the contact layer 105. Rows may be staggered in some examples. The stagger may be characterized by an orientation relative to an edge or other reference line associated with the contact layer 105. For example, the stagger may be characterized by an angle A between a midline 210 of the contact layer 105 and a line through the midpoints of the apertures 125 in adjacent rows parallel to the edges 205. The angle A may vary. For example, a stagger of about 45 degrees or about 60 degrees may be suitable for some embodiments. A pattern may also be characterized by a pitch P, which indicates the spacing between the centers of apertures. Some patterns may be characterized by a single pitch value; others may be characterized by at least two pitch values. For example, if the spacing between centers of the apertures 125 is the same in all orientations, the pitch P may be characterized by a single value indicating the diagonal spacing between centers of the apertures 125 in adjacent rows.

The contact layer 105 may also be characterized by an open area, which can be formed by the apertures 125. The open area may be expressed as a percentage of an area defined by edges of the contact layer 105, such as the area defined by the edges 205 in the example of FIG. 2. An open area of about 40 percent to about 50 percent of the area of the contact layer 105 may be suitable for some examples. The open area can be used to change the bonding properties of the cover 100, for example.

As illustrated in the example of FIG. 2, some embodiments of the contact layer 105 may additionally have a plurality of apertures 215. The apertures 215 may be characterized by various properties, such as hole shape, hole size, hole pattern, and pattern orientation. In FIG. 2, the apertures 215 may be characterized as slots, for example. The size of slots generally may be characterized by a length, which may be specified as a “cut length.” A cut length of about 2 millimeters may be suitable for some examples. The apertures 215 of FIG. 2 are arranged in a linear pattern in which all of the apertures 215 are aligned parallel to one of the edges 205. In general, the material between each of the apertures 215 may be referred to as a bridge or tie. The linear pattern of apertures 215 in the example of FIG. 2 may be characterized by the spacing between the apertures 215, which may be referred to as a bridge or tie length. A tie length of about 1 millimeter may be suitable for some examples. As illustrated in the example of FIG. 2, the midline of the apertures 215 may be aligned with a midline of a row of the apertures 125. More specifically, in some examples, the midline of the apertures 215 may be aligned with a peripheral row of the apertures 125 that is parallel to the shorter of the edges 205. Peripheral rows are generally characterized as rows of the apertures 125 that are closest to the edges 205, exclusive of rows in which some or all of the apertures 125 are exposed or partially open on the edges 205. In some embodiments, the midline of the apertures 215 may be aligned with interior rows, which are interior to the peripheral rows.

As illustrated in the example of FIG. 3, the apertures 125 may be circular holes having a width D. A width D of about 7 millimeters may be suitable for some examples. FIG. 3 further illustrates an example in which pitch is specified by two values, p1 and p2, indicating the center spacing of the apertures 125 in aligned rows orthogonal to the edges 205. If p1 and p2 are not equal, then p1 is indicative of the shorter pitch. In the example of FIG. 3, p1 may be about 9.8 millimeters and p2 may be about 17 millimeters. The example values of D, p1, and p2 forms an open area of about 46 percent of the area defined by the edges 205 of FIG. 2.

FIG. 4 is an isometric view of an example of the cover 100, illustrating additional details that may be associated with some embodiments. In FIG. 4, the apertures 130 in the shell layer 110 and the apertures 215 (not visible in FIG. 4) in the contact layer 105 are aligned and define one or more sacrificial segments 405. Each of the handling bars 115 may be coupled to one of the sacrificial segments 405. For example, each of the handling bars 115 may be at least partially laminated or otherwise disposed between the contact layer 105 and the shell layer 110. In some examples, an interior edge of the handling bars 115 may be exterior to the apertures 130 and the apertures 215, and the handling bars 115 may extend past the edges of the contact layer 105 and the shell layer 110.

In some embodiments, the shell layer 110 and the contact layer 105 may be coextensive. The release liner 120 may be coextensive with the contact layer 105, and may extend past the contact layer 105 to coincide with exterior edges of the handling bars 115.

The release liner 120 may be configured to protect the contact layer 105 and any adhesive prior to use. The release liner may be embossed in some examples. The release liner 120 may comprise two or more release panels in some embodiments. For example, the release liner 120 may comprise one or more panels that can be positioned along opposing edges of the contact layer 105. A first release panel may overlap or otherwise extend over a portion of a second release panel in some embodiments. In other embodiments, the release liner 120 may additionally have a third release panel, which can be overlap or otherwise extend over a portion of at least one of the other release panels. In some embodiments, the release liner 120 may have the same size as the contact layer 105. The release liner 120 may also have one or more release tabs, which may be integral to or otherwise coupled to one or more release panels in some embodiments.

As used herein, “release liner” generally means an apparatus for protecting a surface, which may prevent premature exposure and/or contamination of the surface. A release liner may be particularly advantageous for protecting a surface that is tacky, sticky, or otherwise adherent, such as a surface with an adhesive. For example, a release liner can protect an adhesive surface. A release liner can hold to the surface until time or conditions for release, but may also be removed under the right conditions to allow the surface to be used at the appropriate time.

The release liner 120 (or one or more release panels) may comprise or consist essentially of a casting paper or a polymer film, for example. In some embodiments, the release liner 120 may comprise or consist of a polyethylene film. Further, in some embodiments, the release liner 120 may be a polyester material such as polyethylene terephthalate (PET) or similar polar semi-crystalline polymer. The use of a polar semi-crystalline polymer for the release liner 120 may substantially preclude wrinkling or other deformation of the cover 100. For example, a polar semi-crystalline polymer may be highly orientated and resistant to softening, swelling, or other deformation that may occur when brought into contact with components of the cover 100, or when subjected to temperature or environmental variations, or sterilization.

Further, a release agent may be disposed on a side of the release liner 120 that is configured to contact the contact layer 105. As used herein, a “release agent” generally means a material or substance that provides a release effect with respect to the protected surface. A release agent enables a release liner to perform with respect to the relevant surface. For example, the release agent may be a silicone coating and may have a release factor suitable to facilitate removal of the release liner 120 by hand and without damaging or deforming the cover 100. In some embodiments, the release agent may be a fluorocarbon or a fluorosilicone, for example. In other embodiments, the release liner 120 may be uncoated or otherwise used without a separate release agent. For example, the properties of the release liner material may essentially serve as a release agent.

In some embodiments, the cover 100 may comprise two or more adhesives, and the release liner 120 may comprise two or more corresponding release agents. For example, the contact layer 110 and the shell layer 110 may have different adhesive peel strengths, and the release liner 120 may be configured with more than one release agent configured for different peel strengths.

Some embodiments of the release liner 120 may comprise or consist essentially of a first release agent adapted for the first adhesive of the cover 100, and a second release agent adapted for the second adhesive of the cover 100. The first release agent differs from the second release agent in some embodiments. For example, the first release agent and the second release agent may comprise different materials, which can include different chemicals and/or different concentrations of the same chemical. The first and second release agents can also differ based on surface texture or topography, for example. In some embodiments, the first release agent may be configured to interact with a first area of the cover 100 having the first adhesive, such as the shell layer 110, and the second release agent may be configured to interact with a second area of the cover 100 having the second adhesive, such as the contact layer 105.

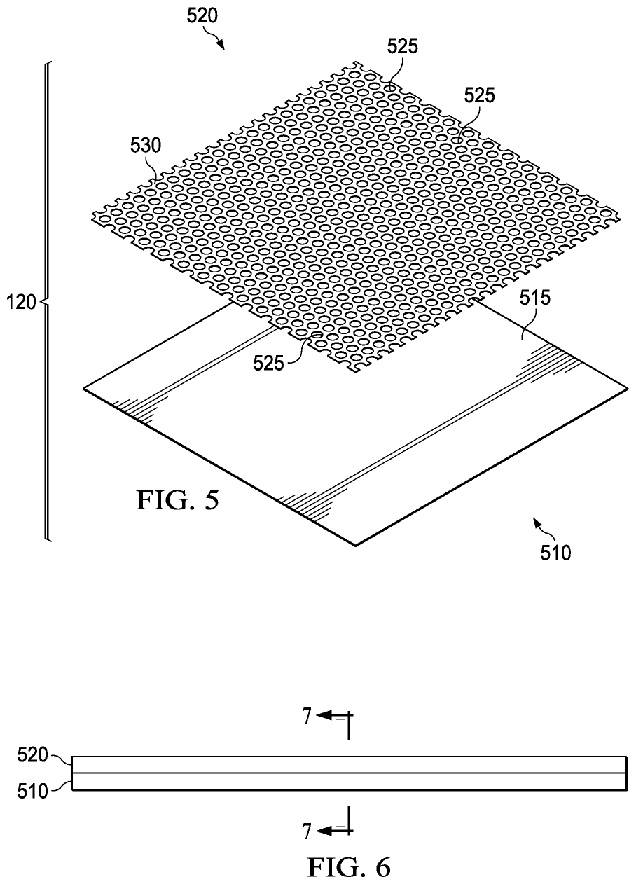

FIG. 5 is an exploded view of an example of the release liner 120, illustrating additional details that may be associated with some embodiments. For example, the release liner 120 may have more than one layer as illustrated in FIG. 5. More particularly, the release liner 120 of FIG. 5 comprises a first layer 510 and a second layer 520. The first layer 510 comprises a first release agent 515 and the second layer 520 comprises a second release agent 530. The second layer 520 typically also comprises a plurality of perforations 525. For example, the perforations 525 may be through-holes passing from one planar surface to the other planar surface of second layer 520. The second release agent 530 can span substantially an entire planar surface of the second layer 520. In some embodiments, the perforations 525 may be configured to align with the apertures 125 of the contact layer 105. For example, the plurality of perforations 525 may be positioned and oriented in the second layer 520 in a pattern matching the apertures 125 in the contact layer 105, and each of the perforations 525 may be sized to be at least as large as each of the apertures 125 in the contact layer 105 corresponding to the perforations 525. The second layer 520 may be disposed adjacent to the first release agent 515 such that the first release agent 515 is exposed through the plurality of perforations 525 in the second layer 520. For example, the first release agent 515 may span substantially the entire upper surface of the first layer 510, or the first release agent 515 may form a pattern on the upper surface of the first layer 510 matching the perforations 525 in the second layer 520 so that the first release agent is exposed through the perforations 525 in the second layer when the layers are disposed as shown in FIG. 5. The second release agent 530 may be located on the surface of the second layer 520 opposite the first release agent 515. For example, the second release agent 530 may substantially cover or span the entire surface of the second layer 520. Typically, the first layer 510 is parallel to and/or in contact with the second layer 520. For example, the first layer 510 may be stacked adjacent to the second layer 520 with planar surfaces contacting each other. Although not shown, in some embodiments there may be one or more interposing layers between the first layer 510 and the second layer 520, so long as the interposing layer does not interfere with exposure of the first release agent 515 through the perforations 525. If the second layer 520 with perforations 525 is stacked on the first layer 510, the first release agent 515 may be exposed through the perforations 525 in the second layer 520, while the second release agent 530 may be exposed as an uncovered surface of the second layer 520 opposite the first layer 510.

As illustrated in the example of FIG. 5, the first layer 510 may be adjacent to the second layer 520. The first release agent 515 may be located on a surface of the first layer 510, which may be located between the first layer 510 and the second layer 520, so that the first release agent 515 can be exposed through the perforations 525 in the second layer 520 of the release liner 120. The second release agent 530 may be located on a surface of the second layer 520, opposite the first layer 510 and the first release agent 515, so that the second release agent 530 is also exposed. In the example of FIG. 5, the first release agent 515 and the second release agent 530 are configured to face the same direction or uni-directionally. Accordingly, this configuration of the release liner 120 exposes both the first release agent 515 and the second release agent 530 simultaneously and/or uni-directionally (e.g. upward as shown in FIG. 5). This configuration of the release liner 120 may also expose both the first release agent 515 and the second release agent 530 in a pattern effectively matching the pattern of exposure of the first adhesive of the shell 110 and the second adhesive of the contact layer 105 for the cover 100.

FIG. 6 is a side elevation view of the release liner 120 of FIG. 5, illustrating additional details that may be associated with some embodiments. In FIG. 6, the first layer 510 may be disposed adjacent to the second layer 520. For example, the first layer 510 may be disposed in stacked relationship with the second layer 520, with planar surfaces contacting each other.

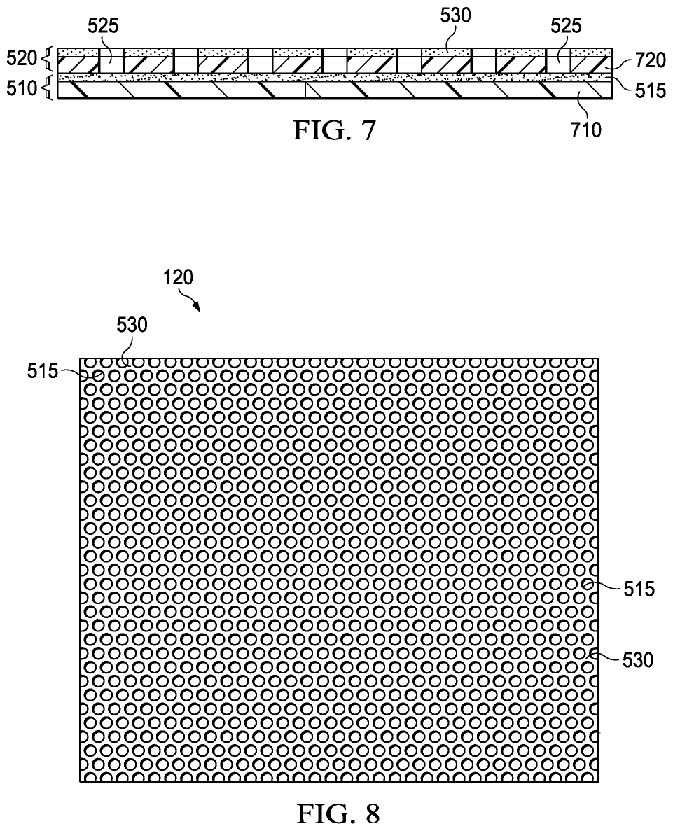

FIG. 7 is a schematic cross-section view of the release liner 120 of FIG. 6, illustrating additional details that may be associated with some embodiments. The perforations 525 of FIG. 7 pass through the thickness of the second layer 520. The first release agent 515 may be located on a surface of the first layer 510 directly adjacent to the second layer 520, so that the first release agent is exposed through the perforations 525 in the second layer 520. The first release agent 515 may be disposed on at least a surface of the first layer 510, and the second release agent 530 may be disposed on at least a surface of the second layer 520.

In FIG. 7, the first layer 510 further comprises a first carrier 710. In some embodiments, the first release agent 515 may be disposed on the carrier 710 of the first layer 510. For example, the first release agent 515 may be a coating disposed on the carrier 710, such that the carrier 710 and the first release agent 515 jointly form the first layer 510. In such configurations, the carrier 710 may be selected for its mechanical characteristics and/or cost, without concern for its release properties, since the release properties of the first layer 510 may typically be governed exclusively by the coating of the first release agent 515. In some embodiments, however, the first layer 510 may not include a separate carrier at all. Instead, the first layer 510 may consist essentially of the first release agent 515. For example, the first release agent 515 can be formed as a film and serve effectively as its own carrier.

The second layer 520 may further comprise a second carrier 720, with the second release agent 530 disposed on the second carrier 720. For example, the second release agent 530 may be a coating disposed on the carrier 720, such that the carrier 720 and the second release agent 530 jointly form the second layer 520. In such configurations, the carrier 720 may be selected for its mechanical characteristics and/or cost, without concern for its release properties, since the release properties of the second layer 520 may typically be governed exclusively by the coating of the second release agent 530. In some embodiments, the second layer 520 may not include a separate carrier at all. Instead, the second layer 520 may consist essentially of the second release agent 530. For example, the second release agent 530 can be formed as a film and serve effectively as its own carrier. So, either the first layer 510 or the second layer 520 may have a carrier, or in some instances, not have a carrier.

In embodiments where either the first or second layer includes a separate carrier, such as the carrier 710 and/or the carrier 720, either carrier may comprise a casting paper, a film, or a polyester material. In some instances, the film may be a polymer film, for example a polyurethane film. In some instances, the polyester material may be a polar semi-crystalline polymer, such as polyethylene terephthalate by way of example. In some embodiments, the release liner 120 may comprise an embossed surface (e.g. configured to facilitate release). In some embodiments, the carrier may comprise a first layer carrier and a second layer carrier, with the carriers of the separate layers jointly serving as an overall carrier of the release liner 120 as a whole.

Some embodiments of the release liner 120 may employ a different configuration, without perforations through the second layer 520 to expose the first release agent of the first layer 510. For example, the first release agent 515 may be disposed on or adjacent to the second release agent 530 in a pattern, such that both the first release agent 515 and the second release agent 530 are presented. For instance, the first release agent 515 may be a coating distributed in a pattern so as to interact with the first adhesive of the cover 100. Typically, such a pattern of the first release agent 515 may not interfere with the ability of the second release agent 530 to interact with the second adhesive of the cover 100.

FIG. 8 is a plan view of another example of the release liner 120, illustrating additional details that may be associated with some embodiments. FIG. 8 illustrates an embodiment of the release liner 120 in which the first release agent 515 and the second release agent 530 may be simultaneously presented for interaction with multiple adhesives, without the need for any exposing perforations. More particularly, the release liner 120 of FIG. 8 comprises a planar surface having a pattern presenting both the first release agent 515 and the second release agent 530. The pattern of the two release agents 515 and 530 may typically be configured to match the pattern of the first and second adhesives in the cover 100. For example, the pattern of the first release agent 515 may substantially match the pattern of the apertures 125 in the contact layer 105, and the pattern of the second release agent 530 may substantially match the bridge pattern of the contact layer 105. In FIG. 8, both the first release agent 515 and the second release agent 530 may face the same direction and/or may be located substantially in the same plane.

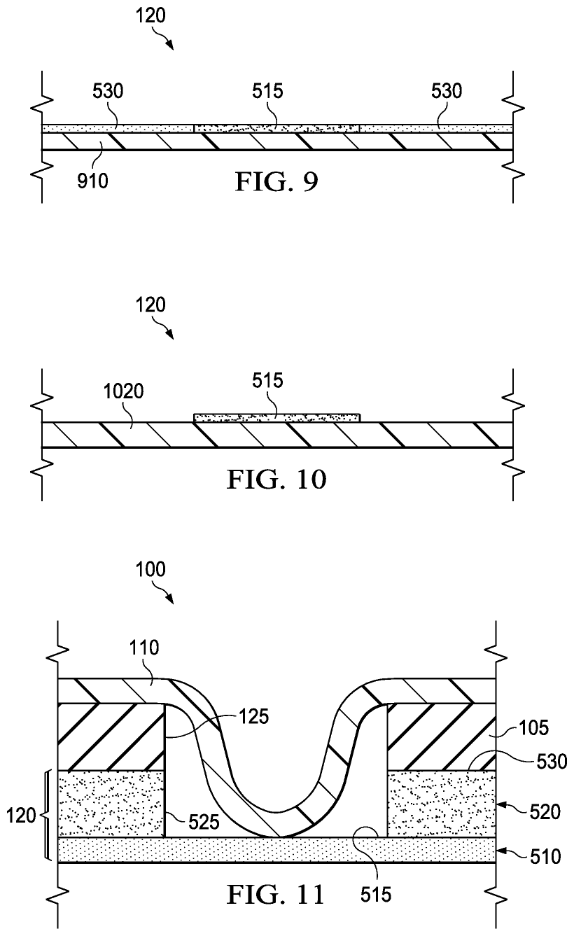

FIG. 9 is a schematic view of an exemplary embodiment of the release liner 120 shown in FIG. 8, illustrating one configuration in which the release agent pattern of FIG. 8 may be achieved. In FIG. 9, the release liner 120 may be formed of a single carrier 910 with both the first release agent 515 and the second release agent 530 disposed thereon. The first release agent 515 and the second release agent 530 may both be disposed on the same surface of the carrier 910, for example as coating in a pattern providing simultaneous and/or uni-directional exposure of both the first release agent 515 and the second release agent 530. In some embodiments, the first release agent 515 may be located adjacent to the second release agent 530, with both the first release agent 515 and the second release agent 530 located in substantially the same plane. For example, the coating pattern may be formed with the two release agents 515 and 530 being co-planar. The coating pattern of the two release agents may be formed by printing of the release agent materials on the carrier 910, for example.

FIG. 10 is a schematic view of another example embodiment of the release liner 120 shown in FIG. 8, illustrating another configuration in which the release agent pattern of FIG. 8 may be achieved. In FIG. 10, the release liner 120 may comprise a carrier 1020 formed of the second release agent, and the first release agent 515 may be a coating disposed in a pattern on the carrier 1020. In FIG. 10, the pattern of the first release agent 515 does not cover the entirety of the surface of the carrier 1020, which can allow both the first release agent 515 and the second release agent 530 to be presented via uncovered portions of the carrier 1020. This type of coating may be accomplished by printing of the first release agent 515 onto the carrier 1020, for example. Alternatively, a uniform coating of the first release agent 515 may be applied to the carrier 1020, and then portions of the first release agent 515 can be removed to form the pattern and expose the second release agent 530. For example, the first release agent 515 may be applied by spraying, rolling, dipping, plasma coating, printing, lamination, etc. An alignment feature such as an index mark (not shown) may be advantageous to ensure that the pattern matches and can later be aligned with the apertures 125 of the contact layer 105 of the corresponding cover 100.

Regardless of the specific approach or embodiment, the first release agent 515 and second release agent 530 may typically differ. For example, the first release agent 515 may be configured to facilitate manual release from acrylic-based adhesive, while the second release agent 530 may be configured to facilitate release from a silicone-based adhesive. Thus, the first release agent 515 may comprise one or more of silicone, fluorocarbon, fluorosilicone, and PTFE, while the second release agent 530 may comprise one or more of acrylic and polyethylene.

FIG. 11 is a detailed schematic view of an example of the cover 100, illustrating additional details that may be associated with some embodiments of the release liner 120 disposed for simultaneous interaction with the contact layer 105 and the shell layer 110. As illustrated in the example of FIG. 11, the release liner 120 may interact with areas of the cover 100 having different types of adhesive. For example, the first release agent 515 may interact with a first area of the cover 100, and the second release agent 530 may interact with a second area of the cover 100. More particularly, in some configurations the first release agent 515 may interact with an area of the first adhesive of the shell 110, and the second release agent 530 may interact with an area of the second adhesive of the contact layer 105. As shown in the example of FIG. 11, the first layer 510 of the release liner 120 can be stacked adjacent to the second layer 520, and may also be adjacent to the shell 110 via exposure through the aligned perforation 525 in the second layer and aperture 125 in the contact layer 105. The second layer 520 of the release liner 120 can be stacked adjacent to the contact layer 105 of the cover 100, opposite the shell 110. The contact layer 105 can be located between the shell 110 and the second layer 520 of the release liner 120. The shell 110 of FIG. 11 can be stacked adjacent to the contact layer 105, opposite the second layer 520 of the release liner 120.

As illustrated in the example of FIG. 11, the apertures 125 and the perforations 525 may be aligned, and portions or areas of the shell 110 may be exposed to the first layer 510 of the release liner 120 through the apertures 125 and perforations 525. Thus, the shell 110 may interact with the first release agent 515 through the apertures 125 and the perforations 525, allowing the first release agent 515 to interact with the adhesive of the first area. The second release agent 530 may be located on the second layer 520 of the release liner 120, so as to be adjacent to the contact layer 105. This may allow the second release agent 530 to interact with the adhesive of the second area.

While the shell 110 is shown in FIG. 11 as protruding through both the aperture 125 and the perforation 525 to the degree that the shell 110 contacts the first layer 510 of the release liner 120, the amount of penetration of the shell 110 through the apertures 125 and the perforations 525 may vary. For example, the shell 110 may have no interaction with the release liner 120 when first assembled, while the shell 110 may have full or complete penetration after handling. Penetration may increase, for example, after Ethylene Oxide sterilization, after cycling of vacuum and heat, and/or after sitting on the shelf for extended periods (such as up to three years) before use. The configuration illustrated in the example of FIG. 11 demonstrates how the release liner 120 may protect against an extreme scenario. For example, in FIG. 11 a first release agent 515, adapted for the first adhesive of the shell 110, is disposed to interact with the first adhesive of the shell 110 in the instance of complete penetration. In some embodiments, the first adhesive of the shell 110 may comprise the attachment device of the shell layer 110. The second release agent 530 may be disposed to interact with the second adhesive of the contact layer 105. For example, the second release agent 530 may have a surface area and pattern matching that of the second area of the second adhesive of the contact layer 105.

In FIG. 11, the first layer 510 of the release liner 120 is adjacent to the second layer 520 of the release liner 120, with the first release agent 515 located on a surface of the first layer 510 facing towards the second layer 520. The second layer 520 of the release liner 120 is adjacent to the contact layer 105, with the second release agent 530 being located on a surface of the second layer 520 facing towards the contact layer 105. The second adhesive of the contact layer 105 can be located on a surface of the contact layer 105 facing towards the second layer 520 of the release liner 120. Thus, the second release agent 530 can be adjacent to the second adhesive. The contact layer 105 of FIG. 11 is adjacent to the shell layer 110, with the first adhesive of the shell layer 110 located on a surface of the shell layer 110 facing towards the contact layer 105. The second layer 520 of release liner 120 may be located between the contact layer 105 and the first layer 510 of the release liner 120, with the first layer 510 of the release liner disposed opposite the contact layer 105. The contact layer 105 may be located between the second layer 520 of the release liner 120 and the shell layer 110, with the shell layer 110 disposed opposite the second layer 520 of the release liner 120. The apertures 125 and the perforations 525 are aligned in FIG. 11, allowing exposure of the shell layer 110 to the first layer 510 of the release liner 120.

In some embodiments, the first area of first adhesive on the shell 110 may be formed by a layer of acrylic adhesive, and the second area of second adhesive on the contact layer 105 may be formed by a layer of silicone having apertures 125. So in some embodiments in which the contact layer 105 comprises or consists essentially of silicone gel, the silicone gel may serve as the second adhesive. In such embodiments, the first release agent 515 may comprise one or more of the following: silicone, fluorosilicone, fluorocarbon, and PTFE; while the second release agent 530 may comprise one or more of the following: acrylic, polyethylene, fluorocarbon, fluorosilicone, and PTFE.

Typically, the contact layer 105 may have an open area of about 40% to about 50% formed by the apertures 125, and the second layer 520 of the release liner 120 may typically match. Each of the apertures 125 may be approximately circular, for example, with each of the apertures 125 having a diameter within a range of approximately 6 millimeters to approximately 8 millimeters. Typically, each of the perforations 525 in the second layer of the release liner 120 is at least as large as the aperture 125 on the contact layer 105 of the cover 100 corresponding to the perforations 525.

Generally, a method of manufacturing some embodiments of the cover 100 may include perforating the contact layer 105 to form apertures 125 in the contact layer 105. The apertures 125 may be also be formed by punching, cutting, or by application of local RF or ultrasonic energy, for example, or by other suitable techniques for forming a hole in the contact layer. The apertures may be arranged in rows in some embodiments. For example, the contact layer may have a first edge and a second edge parallel to the first edge, and the apertures may be arranged in so that at least one row has a midline parallel to the first edge. A second row may have also have a midline parallel to the second edge. At least one handling bar may be disposed at least partially on the contact layer, and a shell layer having an adhesive may be disposed on the contact layer, at least partially overlapping the handling bar. The adhesive may be configured so that at least some of the adhesive is disposed adjacent to at least some of the apertures in the contact layer. The adhesive may bond the shell layer to the contact layer, securing the handling bar to the shell layer and the contact layer. The shell layer and the contact layer may be perforated in a linear pattern along an interior edge of the handling bar to form a sacrificial segment. The linear perforations preferably align with a midline (within an acceptable tolerance) of an outermost row of apertures in the contact layer 105, which can improve the separation of the sacrificial segment and reduce ragged edges. A tolerance between the linear perforations and the edge of the contact layer 105 may additionally or alternatively favor an alignment between the midline and the edge, which can minimize alignment with a tangent of the row of apertures in the contact layer 105. A release liner may be disposed on the contact layer. Alternatively, the release liner may be disposed on the contact layer before perforating the linear pattern. For example, a suitable pressure may be applied to a roller die to cut through the shell layer and the contact layer without perforating the release liner. In some embodiments, a pressure in a range of about 750 pounds per square inch to about 1000 pounds per square inch may be suitable.

A method of manufacturing some embodiments of the release liner 120 for use with the cover 100 may comprise providing the first layer 510 having the first release agent 515 adapted for a first adhesive; providing the second layer 520 having the second release agent 530 adapted for a second adhesive, wherein the second layer 520 has a plurality of perforations 525; and disposing the first layer 510 adjacent with the second layer 520 so that the first release agent 515 may be exposed through the plurality of perforations 525 in the second layer 520. In some embodiments, forming the first layer 510 may be accomplished by applying a coating of the first release agent 515 to a first carrier. In other embodiments, providing a first layer may be accomplished by forming a sheet of the first release agent 515. In some embodiment, forming the second layer 520 may be accomplished by applying a coating of the second release agent 530 to a second carrier, and forming the plurality of perforations 525 through the second layer 520. In other embodiments, providing the second layer 520 may comprise forming a sheet of the second release agent 530, and forming the plurality of perforations 525 through the second layer 520. In some embodiments, the first layer 510 and the second layer 520 may be attached to form a unitary whole release liner 120. Typically, when perforating the second layer 520 to form a plurality of perforations 525, the perforations 525 may match those of a contact layer 105 comprising the second adhesive. In some embodiments, an alignment feature may be formed on the second layer to facilitate alignment of the perforations 525 with the apertures 125 of the corresponding contact layer 105.

A method of manufacturing some embodiments of the cover 100 for a tissue site may comprise providing the shell layer 110 comprising a first adhesive; providing the contact layer 105 comprising a second adhesive; providing the first layer 510 comprising the first release agent 515 adapted for the first adhesive; providing the second layer 520 comprising a second release agent 530 adapted for the second adhesive; disposing the second layer 520 adjacent to the contact layer 105, for example with the second release agent 530 adjacent to the second adhesive; perforating the contact layer 105 and the second layer 520 of to form the apertures 125 and the perforations 525 in alignment; disposing the shell layer 110 on the contact layer 105 opposite the second layer 520 of release liner 120, for example with the first adhesive facing the contact layer 105 and the first adhesive exposed through the apertures 125; and disposing the first layer 510 on the second layer 520 opposite the contact layer 105. In some embodiments, the contact layer 105 and the second layer 520 of the release liner 120 are perforated simultaneously, for example while they are previously joined or contacting in stacked, parallel plane relationship. This approach may have the benefit of more easily ensuring alignment of the apertures 125 with the perforations 525.

In some embodiments, forming the first layer 510 may be accomplished by applying a coating of the first release agent 515 to a first carrier. In other embodiments, providing a first layer 510 may be accomplished by forming a sheet of the first release agent 515, with the first release agent 515 serving effectively as its own carrier. In some embodiment, forming the second layer 520 may be accomplished by applying a coating of the second release agent 530 to a second carrier. In other embodiments, providing the second layer 520 may comprise forming a sheet of the second release agent 530, with the second release agent 530 serving as its own carrier. So in some embodiments, both the first layer 510 and the second layer 520 of the release liner 120 may be formed by applying a thin film or coating atop a carrier. In some embodiment, both the first layer 510 and the second layer 520 of the release liner 120 may consist essentially of the first release agent and the second release agent respectively, with the layer formed of a sheet of the respective release agent.

In some embodiments, the first layer 510 and the second layer 520 may be attached to form a unitary whole release liner 120. If an adhesive is used to bind the first and second layers of the release liner 120 together, the adhesive may be selected so that the first release agent 515 will not negatively impact such adhesive's effectiveness. In other embodiments, one layer of the release liner 120 may be formed as a coating atop a carrier, while the other layer may consist essentially of the release agent. In such embodiments, one layer may serve as the carrier for the other layer of the release liner. For example, the second layer 520 can be formed to consist essentially of polyethylene, and the first layer 510 of the release liner 120 can be applied to the second layer 520 as a coating.

Typically, when perforating the second layer 520 to form a plurality of perforations 525, the perforations 525 may match those of the contact layer 105. For example, the perforation pattern of the second layer 520 may match the aperture pattern in the contact layer 105. Perforating the contact layer 105 and the second layer 520 of release liner 120 may comprise punching, cutting, or burning the perforations, or other similar techniques.

In use, the release liner 120 may be removed to expose the contact layer 105, which may be placed within, over, on, or otherwise proximate to a tissue site. For example, the contact layer 105 may be centered over a tissue site and a peripheral portion of the contact layer may be applied to an attachment surface adjacent to or proximate to the tissue site. The contact layer 105 may be sufficiently tacky to hold the cover 100 in position, while also allowing the cover 100 to be removed or re-positioned without significant trauma to the tissue site.

The handling bars 115 can facilitate handling the cover 100 until placed, and then the handling bars 115 may be removed. For example, the handling bars 115 of FIG. 4 may be removed by separating the sacrificial segments 405, which may be separated by tearing the contact layer 105 and the shell layer 110 along the apertures 215 and the apertures 130, respectively.

Removing the release liner 120 can also expose adhesive on the shell layer 110 through at least some of the apertures 125. Once the cover 100 is in a desired position, the adhesive may be pressed through the apertures 125 to bond the shell layer 110 to an attachment surface. The apertures 125 at the edges 205 may permit the adhesive to flow around the edges 205, which can enhance the adhesion to an attachment surface.

In some embodiments, the apertures 125 may be sized to control the amount of adhesive exposed through the contact layer 105. In some embodiments, the bond strength of the adhesive may vary in different locations of the cover 100. For example, the adhesive may have a lower bond strength in locations adjacent to apertures that are relatively larger, and may have a higher bond strength where apertures are smaller. Adhesive with lower bond strength in combination with larger apertures may provide a bond comparable to adhesive with higher bond strength in locations having smaller apertures.

The cover 100 can provide a sealed therapeutic environment proximate to a tissue site, substantially isolated from the external environment. The contact layer 105 may provide an effective and reliable seal against challenging anatomical surfaces, such as an elbow or heel, at and around a tissue site. Further, in some embodiments, the cover 100 may re-applied or re-positioned to eliminate creases and other discontinuities in the cover 100 or a tissue site, for example.

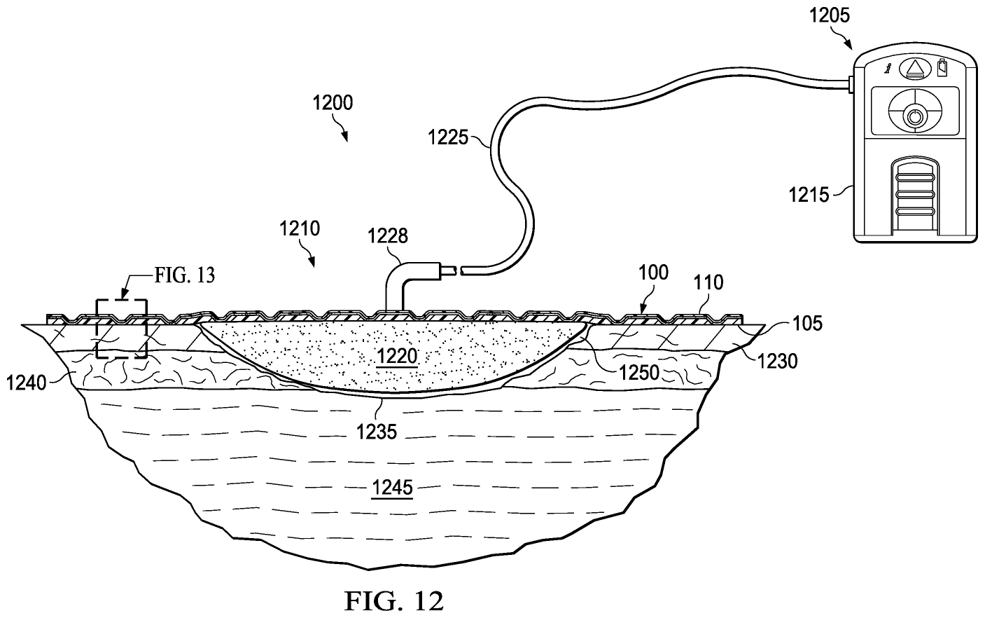

FIG. 12 is a schematic diagram illustrating an example of the cover 100 used with a therapy system 1200 that can reduce pressure in proximity to a tissue site. Clinical studies and practice have shown that reducing pressure in proximity to a tissue site can augment and accelerate growth of new tissue at the tissue site. The applications of this phenomenon are numerous, but it has proven particularly advantageous for treating wounds. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

The therapy system 1200 may include a source or supply of negative pressure, such as a negative-pressure source 1205, and one or more distribution components, such as a dressing or a fluid container. A distribution component is preferably detachable and may be disposable, reusable, or recyclable. A dressing, such as a dressing 1210, and a fluid container, such as a container 1215, are examples of distribution components that may be associated with some examples of the therapy system 1200. As illustrated in the example of FIG. 12, the dressing 1210 may comprise or consist essentially of the cover 100 and a tissue interface 1220.

A fluid conductor 1225 is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface 1228 may facilitate coupling the fluid conductor 1225 to the dressing 1210. For example, such a dressing interface may be a SENSAT.R.A.C.™ Pad available from Kinetic Concepts, Inc. of San Antonio, Tex.

The therapy system 1200 may also include a regulator or controller, and sensors to measure operating parameters and provide feedback signals to the controller indicative of the operating parameters. Some components of the therapy system 1200 may be housed within or used in conjunction with other components, such as sensors, processing units, alarm indicators, memory, databases, software, display devices, or user interfaces that further facilitate therapy. For example, in some embodiments, the negative-pressure source 1205 may be combined with a controller and other components into a therapy unit.

In general, components of the therapy system 1200 may be coupled directly or indirectly. For example, the negative-pressure source 1205 may be directly coupled to the container 1215 and may be indirectly coupled to the dressing 1210 through the container 1215. Coupling may include fluid, mechanical, thermal, electrical, or chemical coupling (such as a chemical bond), or some combination of coupling in some contexts. For example, the negative-pressure source 1205 may be electrically coupled to a controller and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 1205, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 1205 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 1215 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container can reduce waste and costs associated with negative-pressure therapy.

The tissue interface 1220 can be generally adapted to partially or fully contact a tissue site. The tissue interface 1220 may take many forms, and may have many sizes, shapes, or thicknesses, depending on a variety of factors, such as the type of treatment being implemented or the nature and size of a tissue site. For example, the size and shape of the tissue interface 1220 may be adapted to the contours of deep and irregular shaped tissue sites. Any or all of the surfaces of the tissue interface 1220 may have an uneven, coarse, or jagged profile.

In some embodiments, the tissue interface 1220 may comprise or consist essentially of a manifold. A manifold in this context may comprise or consist essentially of a means for collecting or distributing fluid across the tissue interface 1220 under pressure. For example, a manifold may be adapted to receive negative pressure from a source and distribute negative pressure through multiple apertures across the tissue interface 1220, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 1220 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 1220 may also vary according to needs of a prescribed therapy. For example, the tensile strength of foam may be increased for instillation of topical treatment solutions. The 25% compression load deflection of the tissue interface 1220 may be at least 0.35 pounds per square inch, and the 65% compression load deflection may be at least 0.43 pounds per square inch. In some embodiments, the tensile strength of the tissue interface 1220 may be at least 10 pounds per square inch. The tissue interface 1220 may have a tear strength of at least 2.5 pounds per inch. In some embodiments, the tissue interface 1220 may be foam comprised of polyols such as polyester or polyether, isocyanate such as toluene diisocyanate, and polymerization modifiers such as amines and tin compounds. In some examples, the tissue interface 1220 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 1220 may also vary according to needs of a prescribed therapy. For example, the thickness of the tissue interface 1220 may be decreased to reduce tension on peripheral tissue. The thickness of the tissue interface 1220 can also affect the conformability of the tissue interface 1220. In some embodiments, a thickness in a range of about 5 millimeters to 10 millimeters may be suitable.

The tissue interface 1220 may be either hydrophobic or hydrophilic. In an example in which the tissue interface 1220 may be hydrophilic, the tissue interface 1220 may also wick fluid away from a tissue site, while continuing to distribute negative pressure to the tissue site. The wicking properties of the tissue interface 1220 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 1220 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 1220 may further serve as a scaffold for new cell-growth, or a scaffold material may be used in conjunction with the tissue interface 1220 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

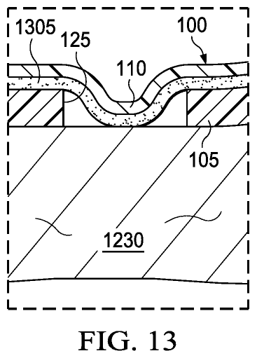

In operation, the tissue interface 1220 may be placed within, over, on, or otherwise proximate to a tissue site. If the tissue site is a wound, for example, the tissue interface 1220 may partially or completely fill the wound, or it may be placed over the wound. The release liner 120 may be removed from the cover 100, and the cover 100 may be placed over the tissue interface 1220 and sealed to an attachment surface near a tissue site. For example, in FIG. 12 the cover 100 may be placed over the tissue interface 1220 and epidermis 1230 peripheral to a tissue site 1235, which extends through the dermis 1240 and into subcutaneous tissue 1245. The contact layer 105 can retain the cover 100 in position, and as illustrated in the detail view of FIG. 13, pressure can be applied to the shell layer 110 to press an adhesive 1305 on the shell layer 110 through the apertures 125 in the contact layer 105 into contact with the epidermis 1230. Thus, the cover 100 can provide a sealed therapeutic environment 1250 proximate to the tissue site 1235, substantially isolated from the external environment, and the negative-pressure source 1205 can reduce pressure in the sealed therapeutic environment 1250.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

Negative pressure applied across the tissue site through the tissue interface 1220 in the sealed therapeutic environment can induce macro-strain and micro-strain in the tissue site. Negative pressure can also remove exudate and other fluid from a tissue site, which can be collected in container 1215.

The systems, apparatuses, and methods described herein may provide significant advantages. For example, the cover 100 can provide a high seal around and over a tissue site, while substantially reducing or eliminating trauma on removal. Additionally or alternatively, the cover 100 may facilitate handling and application to a tissue site. These characteristics may be particularly advantageous for treating wounds with negative-pressure therapy.

The release liner 120 may provide improved performance, particularly with respect to a cover having two different adhesives. For example, the release liner 120 may be tailored to address multiple adhesives. The release liner 120 may also speed development and simplify modifications to such covers 100. For example, the release liner 120 may reduce costs that may be associated with finding a single release agent that may work effectively for more than one adhesive. This may be particularly true for products that may sit on a shelf for extended periods before use, sometimes under excessive heat and/or pressure, which may lead to degradation of the effectiveness of a release liner having only a single release agent. The release liner 120 may have two or more release agents, which can be tailored to the needs of each adhesive type of the cover 100 with no compromise in performance of the release liner 120 for either adhesive type. For instance, this approach may allow selection of each release liner based on known properties of existing release agents, so that prior product history can be relied upon rather than time-consuming testing of interactions with new materials. This may in turn speed development of new configurations having two adhesives, for example by using previously used and time-tested release agents, which each have a known history with respect to a single adhesive.

If something is described as “exemplary” or an “example”, it should be understood that refers to a non-exclusive example. The terms “about” or “approximately” or the like, when used with a number, may mean that specific number, or alternatively, a range in proximity to the specific number as understood by persons of skill in the art field (for example, +/−10%). Use of broader terms such as “comprises”, “includes”, and “having” should be understood to provide support for narrower terms such as “consisting of”, “consisting essentially of”, and “comprised substantially of”. Use of the term “optionally”, “may”, “might”, “possibly”, “could”, “can”, “would”, “should”, “preferably”, “typically”, “often” and the like with respect to any element, component, feature, characteristic, etc. of an embodiment means that the element, component, feature, characteristic, etc. is not required, or alternatively, the element, component, feature, characteristic, etc. is required, both alternatives being within the scope of the embodiment(s). Such element, component, feature, characteristic, etc. may be optionally included in some embodiments, or it may be excluded (e.g. forming alternative embodiments, all of which are included within the scope of disclosure). Section headings used herein are provided for consistency and convenience, and shall not limit or characterize any invention(s) set out in any claims that may issue from this disclosure.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the release liner 120 may be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A release liner for protecting a first adhesive and a second adhesive, the release liner comprising:
a carrier;
a first release agent disposed on the carrier and adapted for the first adhesive; and
a second release agent disposed on the carrier and adapted for the second adhesive.

2. The release liner of claim 1, wherein the carrier comprises a casting paper.

3. The release liner of claim 1, wherein the carrier comprises a film.

4. The release liner of claim 3, wherein the film is a polymer film.

5. The release liner of claim 3, wherein the film is a polyurethane film.

6. The release liner of claim 1, wherein the carrier comprises a polyester material.

7. The release liner of claim 6, wherein the polyester material is a polar semi-crystalline polymer.

8. The release liner of claim 7, wherein the polar semi-crystalline polymer is polyethylene terephthalate.

9. The release liner of claim 1, wherein:
the carrier comprises a first layer carrier and a second layer carrier;
the first release agent is disposed on the first layer carrier;
the second release agent is disposed on the second layer carrier;
the second layer carrier and the second release agent comprise a plurality of perforations; and
the second layer carrier is disposed adjacent to the first release agent such that the first release agent is exposed through the plurality of perforations.

10. The release liner of claim 1, wherein the second release agent has a release factor configured to facilitate manual release from silicone-based adhesive.

11. The release liner of claim 10, wherein the first release agent comprises a fluorocarbon.

12. The release liner of claim 1, wherein the first release agent has a release factor configured to facilitate manual release from acrylic-based adhesive.

13. The release liner of claim 12, wherein the first release agent consists essentially of a coating comprising silicone, fluorocarbon, fluorosilicone, or a combination thereof.

14. A cover for treating a tissue site, the cover comprising:
a first area having a first peel strength;
a second area having a second peel strength; and
a release liner comprising a first release agent adjacent to the first area and a second release agent adjacent to the second area.

15. The cover of claim 14, wherein the first area is formed by a layer of adhesive.

16. The cover of claim 14, wherein the second area is formed by a layer of silicone.

17. The cover of claim 14, wherein:
the first area is formed by a layer of acrylic adhesive;
the second area is formed by a layer of silicone having perforations;
the first release agent comprises silicone; and
the second release agent comprises fluorocarbon.

18. A cover for treating a tissue site, the cover comprising:
a shell layer having an adhesive coating;
a contact layer adjacent to the shell layer, the contact layer comprising a first plurality of apertures; and
a release liner comprising:
a film;
a first release layer disposed adjacent to the contact layer, the first release layer comprising a first release agent and a second plurality of apertures through which at least some of the adhesive coating is exposed; and
a second release layer disposed adjacent to the first release layer, the second release layer comprising a second release agent disposed adjacent to the adhesive coating exposed through the second plurality of apertures.

19. The cover claim 18, wherein the adhesive coating is a pressure-sensitive adhesive.

20. The cover of claim 18, wherein the adhesive coating comprises an acrylic adhesive.

21. The cover of claim 18, wherein the contact layer is formed from a gel.

22. The cover of claim 18, wherein the contact layer is formed from a silicone gel.

23. The cover of claim 18, wherein the first plurality of apertures are circular.

24. The cover of claim 23, wherein the first plurality of apertures have a diameter in a range of about 6 millimeters to 8 millimeters.

25. The cover of claim 23, wherein the first plurality of apertures have a diameter of about 7 millimeters.

26. A release liner for use with a tissue cover having a first adhesive and a second adhesive, wherein the first and second adhesives are different from each other, the release liner comprising:
a first release agent adapted for the first adhesive; and
a second release agent adapted for the second adhesive;
wherein the first release agent is different from the second release agent; and
wherein the first release agent is configured to interact with a first area of the cover having the first adhesive, and the second release agent is configured to interact with a second area of the cover having the second adhesive.

27. The release liner of claim 26, further comprising a first layer and a second layer;
wherein the first layer comprises the first release agent and the second layer comprises the second release agent.

28. The release liner of claim 27, wherein the first layer is parallel and in contact with the second layer.

29. The release line of claims 27-28, wherein the second layer comprises a plurality of perforations.

30. The release liner of claims 27-29, wherein the first layer further comprises a carrier, and wherein the first release agent is disposed on the carrier of the first layer.

31. The release liner of claims 27-30, wherein the second layer further comprises a second layer carrier, and wherein the second release agent is disposed on the second layer carrier.

32. The release liner of claims 27-31, wherein the second layer is disposed adjacent to the first release agent such that the first release agent is exposed through the plurality of perforations in the second layer.

33. The release liner of claims 27-30 and 32, wherein the second layer does not comprise a carrier.

34. The release liner of claims 27-29 and 31-33, wherein the first layer does not comprise a carrier.

35. The release liner of claims 27-34, wherein the first layer is disposed adjacent to the second release agent of the second layer such that both the first and second release agents are exposed.

36. The release liner of claims 30-35, wherein the carrier comprises a casting paper.

37. The release liner of claims 30-36, wherein the carrier comprises a film.

38. The release liner of claim 37, wherein the film is a polymer film.

39. The release liner of claims 37-38, wherein the film is a polyurethane film.

40. The release liner of claims 30-35, wherein the carrier comprises a polyester material.

41. The release liner of claim 40, wherein the polyester material is a polar semi-crystalline polymer.

42. The release liner of claim 41, wherein the polar semi-crystalline polymer is polyethylene terephthalate.

43. The release liner of claims 26-42, further comprising an embossed surface.

44. A cover for treating a tissue site, the cover comprising:
a first area having a first peel strength;
a second area having a second peel strength; and
the release liner of claims 26-43.

45. The cover of claim 44, wherein the first peel strength is greater than the second peel strength.

46. The cover of claims 44-45, wherein:
the first area is formed by a layer of acrylic adhesive;
the second area is formed by a layer of silicone having perforations which expose the first area;
the first release agent has a release factor configured to facilitate manual release from acrylic adhesive; and
the second release agent has a release factor configured to facilitate manual release from silicone.

47. The cover of claims 44-46, wherein the first area is disposed on a shell layer and the second area is disposed on a contact layer which comprises a plurality of apertures;
wherein a pattern of a plurality of apertures on the contact layer matches the pattern of perforations of the second layer of the release liner and each aperture of the contact layer aligns with a corresponding one of the perforations of the second layer;
wherein the first release agent is disposed in proximity to the first area, and the second release agent is disposed in proximity to the second area; and
wherein the contact layer is located between the barrier layer and the second layer of the release liner, such that the first release agent is configured to interact with the first area through the perforations in the second layer and the apertures of the contact layer and the second release agent is configured to interact with the second area.

48. The cover of claims 44-47, wherein:
the first area is formed by a layer of acrylic adhesive;
the second area is formed by a layer of silicone having perforations;
the first release agent comprises one or more of the following: silicone, fluorocarbon, and fluorosilicone; and
the second release agent comprises one or more of the following: acrylic, polyethylene, fluorocarbon, and fluorosilicone.

49. The cover of claims 47-48, wherein the contact layer has an open area of about 40% to about 50% formed by the apertures.

50. The cover of claims 47-49, wherein each of the apertures are approximately circular.

51. The cover of claim 50, wherein each of the apertures has a diameter within a range of approximately 6 mm to approximately 8 mm.

52. The cover of claims 47-51, wherein each of the perforations in the second layer of the release liner is at least as large as the corresponding aperture on the contact layer.

53. A method of manufacturing a release liner for use with a tissue cover having a first adhesive and a second adhesive, the method comprising:
providing a first layer comprising a first release agent adapted for a first adhesive;
providing a second layer comprising a second release agent adapted for a second adhesive, wherein the second layer comprises a plurality of perforations;
disposing the first layer adjacent to the second layer;
wherein the first release agent is exposed through the plurality of perforations in the second layer.

54. The method of claim 53, wherein providing a first layer comprises applying a coating of the first release agent to a first carrier.

55. The method of claim 53, wherein providing a first layer comprises forming a sheet or film of the first release agent.

56. The method of claims 53-55, wherein providing the second layer comprises applying a coating of the second release agent to a second carrier, and forming the plurality of perforations through the second layer.

57. The method of 53-55, wherein providing the second layer comprises forming a sheet or film of the second release agent, and forming the plurality of perforations through the second layer.

58. The method of claims 53-57, further comprising attaching the first layer and the second layer.

59. The method of claims 53-58, further comprising perforating the second layer to form a plurality of perforations.

60. A method of manufacturing a cover for a tissue site, the method comprising:
providing a shell layer comprising a first adhesive;
providing a contact layer comprising a second adhesive;
providing a first layer of release liner comprising a first release agent adapted for the first adhesive;
providing a second layer of release liner comprising a second release agent adapted for the second adhesive;
applying the second layer of release liner to the contact layer;
perforating the contact layer and the second layer of release liner to form a plurality of perforations;
applying the shell layer to the contact layer opposite the second layer of release liner; and
applying the first layer of release liner to the second layer of release liner opposite the contact layer.

61. The method of claim 60, wherein providing a first layer of release liner comprises applying a coating of the first release agent to a first carrier.

62. The method of 60, wherein providing a first layer of release liner comprises forming a sheet or film of the first release agent.

63. The method of 60-62, wherein providing the second layer of release liner comprises applying a coating of the second release agent to a second carrier.

64. The method of claims 60-62, wherein providing the second layer of release liner comprises forming a sheet or film of the second release agent.

65. The method of claims 60-64, further comprising attaching the first layer of release liner and the second layer of release liner.

66. The method of claims 60-65, wherein perforating the contact layer and the second layer of release liner comprises punching, cutting, or burning the perforations.

67. The systems, apparatuses, and methods substantially as described herein.
